(19) United States
(12) Patent Application Publication
(10) Pub. No.: US 2020/0054671 A1
(43) Pub. Date: Feb. 20, 2020

(54) TREATMENT OF ANXIETY DISORDER AND AUGMENTATION OF ANTI-ANXIETY INTERVENTIONS BY ADMINISTRATION OF NOBLE GAS CONTAINING MIXTURES

(71) Applicant: NOBILIS THERAPEUTICS, INC., Portland, OR (US)

(72) Inventors: Vlad BOGIN, Portland, OR (US); Thomas ICHIM, San Diego, CA (US)

(73) Assignee: NOBILIS THERAPEUTICS, INC., Portland, OR (US)

(21) Appl. No.: 16/346,493

(22) PCT Filed: Oct. 31, 2017
(86) PCT No.: PCT/US2017/059387

§ 371 Date: Apr. 30, 2019

Related U.S. Application Data
(60) Provisional application No. 62/414,831, filed on Oct. 31, 2016.

(51) Int. Cl. A61K 33/00; A61P 25/22

(52) U.S. Cl. CPC A61K 33/00; A61P 25/22

(57) ABSTRACT

Disclosed are compositions of matter, treatment methods, and combination therapies for addressing panic attacks in patients suffering from Panic Disorder (PD). In one embodiment the invention provides the use of Noble Gas containing mixtures at a sufficient concentration and frequency to prevent, and/or inhibit frequency of panic attacks. In one particular embodiment xenon gas is administered by inhalation of xenon-oxygen mixtures at concentrations ranging from 15%/85% to 30%/70% with xenon consumption is capped at 3.0 liters per procedure. In other embodiments xenon-oxygen or xenon-oxygen-nitrogen mixtures are administered at sub-anesthetic concentrations which are sufficient to elicit reduction in panic attacks and/or synergize with medications patients are receiving for panic/anxiety symptoms.

### CROSS-REFERENCE TO RELATED APPLICATIONS

The present invention claims priority to U.S. Provisional Application No. 62/414,831, filed Oct. 31, 2016, which is hereby incorporated in its entirety including all tables, figures, and claims.

### FIELD OF THE INVENTION

The invention pertains to the field of psychiatric disorders, more particularly, the invention pertains to the field of panic attacks in patients suffering from panic disorder. Specifically, the invention pertains to utilization of Noble Gases to decrease Panic Disorders.

### BACKGROUND OF THE INVENTION

Panic/anxiety is broadly defined as a state of unwarranted or inappropriate worry often accompanied by restlessness, tension, distraction, irritability and sleep disturbances. This disproportionate response to environmental stimuli can hyperactivate the hypothalamic-pituitary-adrenal axis and the autonomic nervous system, resulting in somatic manifestation of anxiety, including shortness of breath, sweating, nausea, rapid heartbeat and elevated blood pressure (Sanford et al. Pharmacol. Ther. 2000, 88: 197-212). Panic disorders represent a range of conditions and as a result have been classified into multiple distinct conditions, including generalized anxiety disorder (GAD), panic attack, post traumatic stress disorder (PTSD), obsessive compulsive disorder (OCD) and social phobias (Sanford et al. Acta. Psychiatr. Scand. Suppl. 1998, 393: 74-80). Generalized anxiety disorder (GAD) is the most common of the anxiety disorders that is characterized by excessive and persistent worries. In the general population the lifetime prevalence rate of GAD range from 4.1 to 6.6% with somewhat higher rates in woman than in man. The individual with GAD worries about life events such as marital relationships, job performance, health, money and social status. Individuals with GAD startle easily and may suffer from depression. Some of the specific symptoms of GAD include restlessness, motor tension, difficulty concentrating, irritability, and sleep disturbances. The severity of the symptoms over time may be linked to the changing nature of the environmental stressor. With increasing age, GAD symptoms become less severe.

Panic Disorder is a well-studied psychiatric condition that consists of multiple disabling panic attacks characterized by and intense autonomic arousal. In addition, heightened fear and anxiety states occur both during and between panic attacks. Approximately 3% of woman and 1.5% of men have panic attacks. During a panic attack, the individual experiences multiple symptoms including light-headedness, a pounding heart and difficulty in breathing. Panic disorder may be caused by an oversensitive brain system regulating autonomic functions. Potential brain regions involved in panic attack are the locus ceruleus, hippocampus and amygdala. Pathophysiology in the brain GABA-benzodiazepine receptor system may also contribute to the production of panic attack.

### SUMMARY OF THE INVENTION

Various aspects of the invention relating to the above are enumerated in the following paragraphs:

Aspect 1. A composition useful for prevention of panic attacks in a patient suffering from panic disorder consisting of: a) a Noble Gas; b) a gas mixture containing oxygen; and c) optionally a gas mixture containing nitrogen or air.

Aspect 2. The composition of aspect 1, wherein said Noble Gas is selected from a group comprising of: a) helium; b) neon; c) argon; d) xenon; and e) krypton.

Aspect 3. The composition of aspect 2, wherein said Noble Gas comprises said composition at an amount which does not induces anesthesia.

Aspect 4. The composition of aspect 2, wherein said composition comprises 5-40% xenon gas.

Aspect 5. The composition of aspect 4, wherein said xenon gas is admixed with air.

Aspect 6. The composition of aspect 4, wherein said xenon gas is admixed with oxygen.

Aspect 7. The composition of aspect 4, wherein said xenon gas is admixed with an oxygen/nitrogen mixture.

Aspect 8. The composition of aspect 1, wherein administration is performed at a concentration and frequency to induce reduction in TNF-alpha production from brain microglial cells in patients suffering from panic disorder possessing elevated spontaneous production of TNF-alpha as compared to healthy control.

Aspect 9. The composition of aspect 1, wherein administration is performed at a concentration and frequency to induce reduction in TNF-alpha production from brain microglial cells in patients suffering from panic disorder possessing elevated induced production of TNF-alpha as compared to healthy control.

Aspect 10. The composition of aspect 9, wherein said induced production of TNF-alpha occurs as a result of a neural cellular injury.

Aspect 11. The composition of aspect 10, wherein said neural cellular injury is a result of trauma to neural tissue.

Aspect 12. The composition of aspect 10, wherein said trauma to said neural tissue is excitotoxicity.

Aspect 13. The composition of aspect 10, wherein said trauma to said neural tissue is inflammation.

Aspect 14. The composition of aspect 10, wherein said trauma to said neural tissue is degeneration.

Aspect 15. The composition of aspect 10, wherein said trauma to said neural tissue is associated with reduction of proliferation of dentate gyrus cells.

Aspect 16. The composition of aspect 10, wherein said trauma to said neural tissue is associated with reduction of proliferation of subventricular zone cells.

Aspect 17. The composition of aspect 1, wherein administration is performed between 1-7 times per week.

Aspect 18. The composition of aspect 1, wherein administration is performed in a volume of 1-10 liters per treatment.

Aspect 19. The composition of aspect 1, wherein administration is performed in an escalating manner, said escalation involving increasing frequency of administration.

Aspect 20. The composition of aspect 1, wherein administration is performed in an escalating manner, said escalation involving increasing dosage of administration.

Aspect 21. The composition of aspect 1, wherein administration dose and frequency is guided by reduction in patient panic attacks.

Aspect 22. The composition of aspect 1, wherein administration dose and frequency is guided by reduction in patient production of inflammatory cytokines.

Aspect 23. The composition of aspect 1, wherein said inflammatory cytokines are selected from a group comprising of: a) IL-1, b) IL-2; c) IL-6; d) IL-8; f) IL-12; g) IL-15; h) IL-17; and i) IL-33.

Aspect 24. The composition of aspect 23, wherein said inflammatory cytokines are found in the cerebral spinal fluid.

Aspect 24. The composition of aspect 23, wherein said inflammatory cytokines are found in systemic circulation.

Aspect 25. The composition of aspect 23, wherein said inflammatory cytokines produced from patient cells after stimulation with an innate stimulatory molecule.

Aspect 26. The composition of aspect 25, wherein innate stimulatory molecule is selected from a group comprising of: a) a toll like receptor antagonist; b) a damage associated molecular pattern; and c) an excitotoxic agent.

Aspect 27. A method of reducing the number of panic attacks in a patient suffering from panic disorder comprising the steps of: a) selecting a patient in need of therapy; b) administering to said patient a composition containing at least one Noble Gas; c) assessing effects of said therapy and adjusting concentration of said Noble Gas according to said therapeutic response.

Aspect 28. The method of aspect 27, wherein said Noble Gas containing composition contains a gas selected from a group comprising of: a) helium; b) neon; c) argon; d) xenon; and e) krypton.

Aspect 29. The method of aspect 28, wherein said Noble Gas is xenon.

Aspect 30. The method of aspect 29, wherein said xenon is administered at a concentration of 5%-40% diluted with air.

Aspect 31. The method of aspect 29, wherein said xenon is administered at a concentration of 5%-40% diluted with oxygen.

Aspect 32. The method of aspect 29, wherein said xenon is administered at a concentration of 5%-40% diluted with oxygen-nitrogen mixture.

Aspect 33. The method of aspect 27, wherein said composition is administered in a volume of 0.5-10 liters per treatment.

Aspect 34. The method of aspect 27, wherein said composition is administered in a volume of 3-7 liters per treatment.

Aspect 35. The method of aspect 27, wherein administration of said composition occurs between once per day to once every two weeks.

Aspect 36. The method of aspect 27, wherein said administration of said composition occurs as the patient feels the onset on a panic attack.

Aspect 37. The method of aspect 27, wherein said administration of said composition is performed at a concentration and frequency to reduce neuroinflammation.

Aspect 38. The method of aspect 27, wherein said administration of said composition is performed at a concentration and frequency to reduce systemic inflammation.

Aspect 39. The composition of aspect 1, wherein said composition is administered to enhance efficacy of existing therapies for panic attacks.

Aspect 40. The method of aspect 27, wherein administration of said Noble Gas composition is performed to enhance efficacy of existing therapies for panic attacks.

Aspect 41. A method of treating anxiety comprising the steps of: a) selecting a patient in need of therapy; b) administering to said patient a composition containing at least one Noble Gas; c) assessing effects of said therapy and adjusting concentration of said Noble Gas according to said therapeutic response.

Aspect 42. The method of aspect 41, wherein said anxiety is generalized anxiety disorder.

Aspect 43. The method of aspect 41, wherein said anxiety is recurring subclinical anxiety.

Aspect 44. The method of aspect 41, wherein said anxiety is chronic subclinical anxiety.

Aspect 45. The method of aspect 41, wherein said anxiety is persistent anxiety.

Aspect 46. The method of aspect 41, wherein said anxiety is anxious depression.

Aspect 47. The method of aspect 41, wherein said anxiety is neurosis.

Aspect 48. The method of aspect 41, wherein said anxiety is healing avoidance anxiety.

Aspect 49. The method of aspect 41, wherein said anxiety is dissociative anxiety.

Aspect 50. The method of aspect 41, wherein said anxiety is a phobia.

Aspect 51. The method of aspect 50, wherein said phobia is an animal phobia.

Aspect 52. The method of aspect 50, wherein said phobia is a social phobia.

Aspect 53. The method of aspect 50, wherein said phobia is a height anxiety.

Aspect 54. The method of aspect 50, wherein said phobia is claustrophobia.

Aspect 55. The method of aspect 50, wherein said phobia is agoraphobia.

Aspect 56. The method of aspect 49, wherein said dissociative anxiety is associated with flashbacks.

Aspect 57. The method of aspect 49, wherein said dissociative anxiety is associated with depersonalization.

Aspect 58. The method of aspect 49, wherein said dissociative anxiety is associated with derealisation.

Aspect 59. The method of aspect 49, wherein said dissociative anxiety is associated with intrusive thoughts.

Aspect 60. The method of aspect 41, wherein said Noble Gas containing composition contains a gas selected from a group comprising of: a) helium; b) neon; c) argon; d) xenon; and e) krypton.

Aspect 61. The method of aspect 41, wherein said Noble Gas is xenon.

Aspect 62. The method of aspect 41, wherein said xenon is administered at a concentration of 5%-40% diluted with air.

Aspect 63. The method of aspect 41, wherein said xenon is administered at a concentration of 5%-40% diluted with oxygen.

Aspect 64. The method of aspect 41, wherein said xenon is administered at a concentration of 5%-40% diluted with oxygen-nitrogen mixture.

Aspect 65. The method of aspect 41, wherein said composition is administered in a volume of 0.5-10 liters per treatment.

Aspect 66. The method of aspect 41, wherein said composition is administered in a volume of 3-7 liters per treatment.

Aspect 67. The method of aspect 41, wherein administration of said composition occurs between once per day to once every two weeks.

Aspect 68. The method of aspect 41, wherein said administration of said composition occurs as the patient feels the onset on a panic attack.

Aspect 69. The method of aspect 41, wherein said administration of said composition is performed at a concentration and frequency to reduce neuroinflammation.

Aspect 70. The method of aspect 41, wherein said administration of said composition is performed at a concentration and frequency to reduce systemic inflammation.

Aspect 71. The composition of aspect 41, wherein said composition is administered to enhance efficacy of existing therapies for panic attacks.

Aspect 72. The method of aspect 41, wherein administration of said Noble Gas composition is performed to enhance efficacy of existing therapies for panic attacks.

### BRIEF DESCRIPTION OF THE DRAWINGS

FIG. 1 is a bar graph showing changes in HADS_T Scale (subscale «anxiety»)

FIG. 2 is a bar graph showing descriptive statistics for the Zung Self-Rating Anxiety Scale (SAS) and changes compared with baseline (V1) for the evaluation visits and patient groups

FIG. 3 is a bar graph showing changes in HADS D Scale (subscale “depression”)

### DESCRIPTION OF THE INVENTION

The invention provides means of utilizing Noble Gas composition administered alone or as an adjuvant therapy to reduce panic attacks and/or generalized anxiety.

The fundamental finding, which to our knowledge is new, useful and non-obvious, is that administration of various concentrations of xenon gas containing gaseous compositions to patients suffering from panic disorders was associated with reduction of panic attacks, suppression of depressive behavior, and a post-treatment therapeutic effect.

In one embodiment of the invention, patients suffering from panic disorder are treatment by administration of Noble Gas containing compositions. Particularly, administration may be performed by inhalation of the gas, systemic administration, topical administration or administration into a bodily cavity. In a preferred embodiment administration of Noble Gas containing compositions is performed by inhalation several times per week of sub-anesthetic concentrations of Noble Gases, particularly of xenon gas. In one embodiment of the invention, the use of Noble Gases is performed to treatment bipolar disorder. In another embodiment the use of Noble Gases is disclosed for the treatment of depression.

Despite the fact that SSRIs, SNRIs and benzodiazepines have proven efficacy in the treatment of PD, the delayed onset of action for the former and the side effects and the risk of dependence for the latter limit its use in the most active cohort of patients with PD. In addition, there are currently insufficient data on the treatment of refractory PD and on effective augmentation strategies, as well as on the treatment PD with comorbid mental illnesses.

The inert gas xenon was first shown to possess anesthetic properties over 50 years ago[1]. Over the last ten years the interest in xenon as an inhalational anesthetic has increased due to several characteristics associated with its use: cardiovascular stability, rapid induction and emergence from anesthesia, and its analgesic effects—all of which make it an ideal anesthetic [2]. As a result, xenon has become more routinely used as an anesthetic agent in Europe and Japan and has garnered increasing interest in the United States although, primarily due to the higher cost of xenon as compared to other inhalational anesthetics, it has not yet received FDA approval. Evidence suggests that xenon's biological effects may be mediated through its ability to potently block the NMDA receptors [3].

Furthermore, xenon has distinct advantages over other NMDA antagonists, such as ketamine, for future translation to the clinical setting. First, subsedative concentrations of xenon that would sufficiently block the NMDA receptor without producing anesthesia could potentially be administered briefly in a safe and effective manner in the outpatient setting with minimal medical monitoring. Second, in contrast to existing NMDA receptor blockers like ketamine, xenon has been shown to inhibit NMDA receptor activity through competitive inhibition of the co-agonist glycine at the glycine site of the NMDA receptor [3]—a mechanism devoid of psychotomimetic effects.

In one embodiment of the invention, administration of Noble Gas containing compositions is performed with currently available drugs used in the treatment of panic disorder. A number of drugs have either been developed or are being developed for treating the different subclasses of anxiety. Some of these agents such as tricyclic antidepressants and b-adrenoreceptor antagonists found either limited use in treating specific disorders such as performance anxiety b-adrenoreceptor antagonists suppression of the sympathetic manifestations of anxiety) or have fallen out of favor for reasons of efficacy and/or safety. Currently, direct and indirect serotonin receptor agonists [e.g., selective serotonin reuptake inhibitors (SSRI) and buspirone] and benzodiazepines are most often prescribed for treating anxiety disorders with benzodiazepine receptor agonist being a preferred therapeutic modality. See Atack et al. Curr. Drug Targets. CNS. Neurol. Disord. 2003, 2: 213-232; Stahl et al. J. Clin. Psychiatry 2002, 63: 756-757; Uhlenhuth et al. J. Clin. Psychopharmacol. 1999, 19: 23S-24S; Varia et al. Int. Clin. Psychopharmacol. 2002, 17: 103-107; Vaswani et al. Prog. Neuropsychopharmacol. Biol. Psychiatry 2003, 27: 85-102. The ability of benzodiazepines to enhance g-aminobutyric acid (GABA) neurotransmission safely and rapidly is central to their effectiveness in treating anxiety disorder, especially GAD and panic disorders (Stahl et al. J. Clin. Psychiatry 2002, 63: 756-757). Benzodiazepines act by positively modulating the inhibitory neurotransmitter GABA through an allosteric site on the GABA A receptor complex, a ligand-gated chloride ion channel. Nonetheless, the use of benzodiazepines is limited by side effects associated with enhanced GABAergic neurotransmission, manifesting as sedation, muscle relaxation, amnesia and ataxia. Moreover, the potential for abuse and physical dependence is associated with the long-term use of benzodiazepines. Furthermore, some forms of anxiety such as OCD are relatively resistant to benzodiazepine treatment. These therapeutic limitations and the societal burdens of anxiety provide the impetus for the development of novel anxiolytics or anxioselective agents, which in one embodiment of the invention, the therapeutic efficacy of these approaches is augmented by the co-administration of Noble Gas containing mixtures, with a preferred embodiment being administration of xenon at concentrations that are subanesthetic.

In one embodiment of the invention, when using xenon as an acute treatment of PD, reduction in both frequency and severity of panic attacks and anxiety level is observed during the first three treatment sessions, and by the end of treatment the vast number of patients experienced complete resolution of panic attacks while anxiety symptoms decreased to a subclinical level.

In another embodiment, xenon is administered, as described in the Example Section as a means of augmenting efficacy of therapy in patients suffering from comorbidities. In another embodiment, the unique observation that after treatment of both “pure” PD and PD with psychiatric comorbidities, anxiolytic effect of xenon was maintained for at least six months, which clinically manifested by cessation of panic attacks is disclosed.

In another embodiment, xenon administration, or administration of Noble Gas containing compositions is performed as a monotherapy, or in combination with pharmacotherapy, and/or in combination with cognitive-behavioral therapy (CBT) [7]. At the same time, in clinical practice, the use of CBT in patients with severe PD in the early stages is often hampered by the high intensity of the somatic symptoms of anxiety and hypochondriacal fears, for relief of which providers often resort to the use of benzodiazepines. This approach can affect cognitive function and impede effective personal involvement in psychotherapy. In this sense, according to the current invention, xenon treatment may be a good alternative to benzodiazepine tranquilizers because of its good tolerability, rapidity of onset and its lack of addictive potential.

The main methodological limitation of this study is its open design. In order to determine the place xenon in the treatment of PD additional randomized, placebo-controlled clinical trials of xenon and psychotropic substances used for the treatment of PD (SSRIs, SNRIs, benzodiazepines) are needed. The design of such studies should distinguish between direct anxiolytic effect of xenon and a potential placebo effect.

In the practice of the invention, xenon is one of the preferred Noble gases for administration, in part because of its blood/gas partition coefficient being lower than other inhalational agents used in medicine. It is important to note, however, that despite it's fast onset of effect the half-time for equilibration between xenon concentration in arterial blood and at the site of action is measured to be 1.49±0.04 minutes versus 3.91±0.1 minutes. Successful loading of xenon in the brain during inhalational anesthesia is observed after approximately 15 minutes although the end-expiratory xenon concentration reached a plateau after 7 minutes. Thus cerebral xenon uptake rate is only moderate, xenon's fast onset of action being largely due to its extremely fast alveolar uptake [4].

### Example: Clinical Trial Demonstrating Efficacy of Xenon in Panic Attack Reduction

Patients

This investigator-initiated study was performed under a prospective clinical trial protocol approved by the Institute of Mental Health and Addictology, which is accredited by the Ministry of Health of the Russian Federation to conduct clinical trials (#57689). Study conduct was in compliance with all ethical standards and good clinical practice. Ninety outpatients with a diagnosis of “panic disorder» (F41.0) according to ICD-10 were enrolled through the Institute of Mental Health and Addictology. Five patients dropped out of the study due to minor side effects, predominantly lightheadedness and headaches, and 4 patients dropped out of the study for unspecified reasons. As the intention to treat analysis was not utilized due to the open label design of the study, 81 patients with PD (49 women and 32 men), mean age was 35.2 years (range −18-69) were studied. Patients were randomized into 2 groups: with “pure” PD (group 1) and “comorbid” PD, when it was co-diagnosed with other mental illnesses (group 2). All patients with isolated PD (Group 1, n=42) received monotherapy with xenon at the aforementioned schedule, while the majority of patients (94.9%) with PD and comorbid conditions, which were predominantly depression (group 2, n=39), in addition to xenon administration continued treatment for comorbid psychiatric disorders, which mainly consisted of antidepressants (SSRIs and SNRIs). In these patients, the reason for xenon treatment was the increase in the frequency and severity of panic attacks despite ongoing treatment with stable pharmacotherapy of at least 3 to 6 months' duration.

Xenon Administration

Administration of xenon was performed through inhalation of xenon-oxygen mixtures that were escalated from 15%/85% to 30%/70% with titration increments of 5% per session. Each patient in the study underwent between 6 and 7 treatments with xenon-oxygen mixture. The first three sessions were carried out daily and from session 4 onward—every other day. The selected dosing regime and the composition of the gas mixtures were based on the historical evidence of safety of subanesthetic use of xenon in imaging.

Medical grade xenon (“medksenon»®, 99.9999%, manufacturer: Atommedcenter, Moscow, Russia) and medical grade oxygen in separate containers were admixed. Mixing and administration of gases in preset concentration and volume was accomplished with the use of the medical device MAGi-AMTS1, which enables the operator to adjust the concentration of xenon in the gas mixture, and which contains the electronic flow meter with a software module that allows for such adjustments. Administration of xenon-oxygen mixture to the patient was carried out via a face mask. Patients were asked to slowly inhale, holding breath for 5 to 10 seconds; exhale into the loop and after 35-40 seconds exhale outside the contour and breath in the new portion of gas mixture. Xenon inhalation lasted from 2.5 to 4 minutes, and the xenon consumption was capped at 3.0 liters per procedure. The patients were assessed subjectively by the provider, while the vital signs (pulse, blood pressure, oxygen saturation) were continuously monitored.

Patient Assessment

Patients were evaluated after each xenon inhalation and at 30 and 180 days after completion of treatment. To this end, we employed clinical psychopathological and clinical catamnestic methods, and psychometric scales that are widely used internationally to assess the treatment of mental disorders. Scale Assessment, Zung Self-Rating Anxiety Scale (SAS) was performed prior to starting therapy (V1), and at 1 and 6 months after treatment. According to this scale, SAS index of less than 45 points corresponds to the normal value, 45-59—to mild-to-moderate degree of anxiety, 60-74—to high degree of anxiety, more than 75—to an extremely high-level of anxiety. Hospital Anxiety and Depression Scale (Hospital Anxiety and Depression Scale, HADS_T-anxiety subscale, HADS_D—Depression subscale) was used prior to the (V1), after the third (V3) and sixth (V6) xenon administrations. Categories for the assessment for each of the following subscales are as follows: 0-7 points—normal (absence of reliable pronounced symptoms of anxiety/depression); 8-10 points—subclinical anxiety/depression; 11 points and above—symptomatic anxiety/depression. Clinical Global Impression Scale (CGI-I—improvement subscale, CGI-S-severity of the disease subscale) was used before treatment and after each of the following 6 xenon treatments (V1, V2, V3, V4, V5, V6).

Statistical analysis of the results was carried out via statistical and analytical methods using Microsoft Excell 2000 program and with Statistica statistical tools (http://www.statsoft.com/, http://www.statsoft.ru/).

The two groups of patients, with “pure” PD (group 1, n=42) and with “comorbid” PD (group 2, n=39) were well matched (Table 1). For patients in group 2 the following comorbid disorders were most commonly observed: mixed anxiety-depressive disorder (43.6%), bipolar affective disorder (10.3%), recurrent depressive disorder (10.3%), obsessive-compulsive disorder (5.1%), and other nonpsychotic mental disorders (12.8%, heading F48).

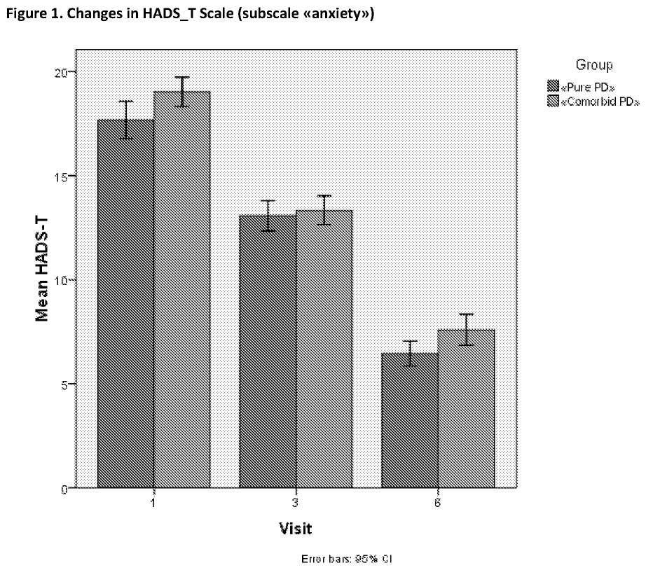

Changes in the subscale of “anxiety” in Hospital Anxiety and Depression Scale (HADS_T) are presented in FIG. 1. The total score on this scale in both groups corresponded to the level of “clinically severe anxiety” (17.7 and 19.0, respectively), and showed a decrease (−4.6 and 5.7 points, respectively) after 3 sessions (V3) of xenon administration (13.3 and 13.3, respectively). By the end of treatment (V6), the overall scores in both groups corresponded to the category of the “norm” for HADS_T Scale. Statistical analysis of the changes in SAS Scale using paired test samples is presented in Table 2.

Analysis of Clinical Global Impression Scale Improvement Subscale (CGI-I) changes after the third treatment shows a more significant improvement with xenon treatment (“marked improvement” on the CGI-I) in group 1 than in group 2 (40.5% and 10.3%, respectively, when compared to baseline). This trend persisted after 6 treatments: the indicator “very much improved” in patients with “pure” PD was 52.4%, while for those with “comorbid” PD it was only 12.8% (Table 3). According to the Clinical Global Impression Scale Severity of the Disease Subscale (CGI-S) (Table 4), before the start of treatment, both groups of patients demonstrated a pronounced degree of impairment: the indicator “significantly pronounced disease” was at 90.5% and 87.2%, respectively. After the third procedure, reduction in the severity of disorders was more pronounced in group 1: the indicator “moderately severe disease” was 48.7% and 11.9%, respectively. At the same time, upon completion of xenon treatments the differences between the two groups disappeared and most patients in both groups reached the “borderline” level (82.1% and 88.1%, respectively).

Thus, by analyzing the changes in the indices of psychometric scales (HADS_T, CGI-I, CGI-S) it can be concluded that the use of xenon treatment in PD produced rapid onset of action, statistically significant clinical improvement, and complete cessation of panic attacks after the 6^{th} treatment.

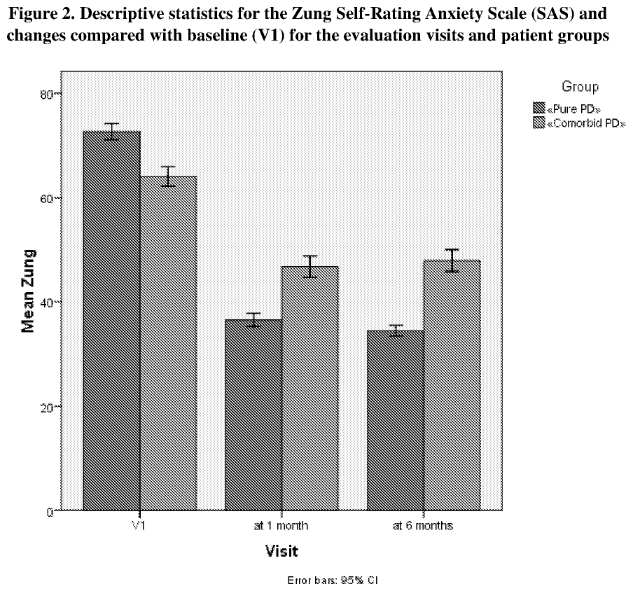

Symptom changes on the SAS scale are presented in FIG. 2. The initial presentation in both groups corresponded to “high level of anxiety” (72.7 and 64.1, respectively). One month after treatment, all patients showed a decrease in the SAS total score, although it was more pronounced in group 1: 36.5 points (which corresponds to “no anxiety”) against 46.8 points in group 2 (“minimum degree of anxiety”). Furthermore, these parameters remained approximately at the same level throughout the study follow up (34.5 and 47.9, respectively). Statistical analysis of changes in SAS scale using paired test samples are presented in Table 5.

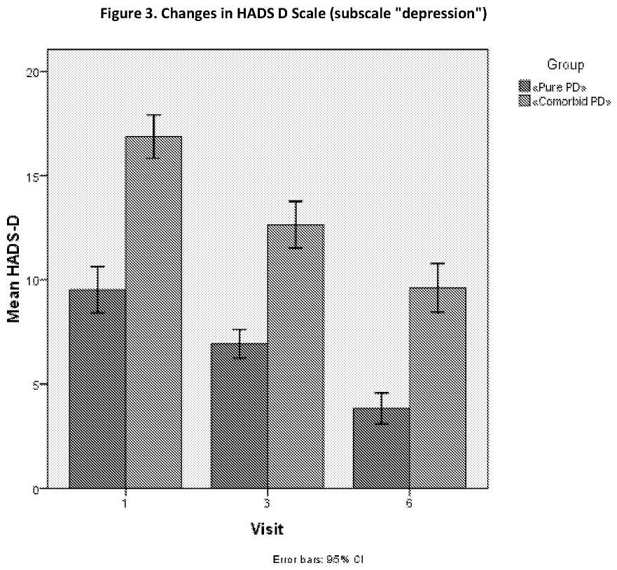

As noted above, in the modern classifications, in addition to “major” episodes that meet the criteria of a panic attack based on the number of symptoms, “limited symptom” (“minor”) panic attacks have been described, which, nevertheless, have an impact on social functioning and quality of life. As seen in FIG. 3, the mean number of “major” panic attacks per month in group 2 was even greater than that of the group 1 (7.7 and 11.7, respectively), while the number of “minor” attacks were slightly higher in group 1, or patients with “pure” PD (44.8 and 41.7, respectively). 6 months after treatment “major” panic attacks were absent in both groups, while “minor” panic attacks occurred in a very few cases (0.3 to 1, respectively).

The results of SAS scales and lack of panic attacks after 6 months of treatment indicate the sustained anxiolytic effect of xenon administration.

As has already been noted, most often encountered comorbid mental conditions in group 2 included depressive disorders. The high degree of severity of depressive symptoms indicate that the traditional in these cases antidepressant therapy was ineffective. While the effect of xenon directly on depression is beyond the scope of this study, the analysis of HADS_T subscale “Depression” of HADS_D scale (Table 9) warrants some observations on this topic. According to HADS_D, “clinically severe depression” was absent in 66.7% of the patients in group 1 before the start of treatment, while it was present in 92.3% of group 2. After 3 xenon treatments it was absent in 90.5% of patients in group 1, but was still present in the majority of patients in Group 2 (82.1%). By the end of the active phase of treatment “clinically severe depression” was negligible in patients of group 1 (2.4%), and it decreased to 46.2% in group 2.

Xenon therapy was generally well tolerated, side effects, mainly headache and dizziness, were rare and lead to only 5 patients dropping out from the study (5.8%). After carefully reviewing the data from these patients, it should be noted that four of them were found to have clinical symptoms of mild organic brain disease of vascular origin (F06.71 heading ICD-10), which is indirectly confirmed by the results of head and neck Doppler ultrasound. It was previously demonstrated that inhalation of xenon can increase cerebral blood flow [6].

TABLE 1
Social and demographic characteristics of the patients

| | | | Group | | |
|---|---|---|---|---|---|
| | | | «Pure PD» | «Comorbid | Total |
| | | | (n = 42) | PD» (n = 39) | (n = 81) |
| Age, years | Mean | | 36.1 | 34.3 | 35.2 |
| | Standard | | 12.90 | 12.20 | 12.52 |
| | deviation | | | | |
| | Median | | 32.0 | 33.0 | 33.0 |
| | Minimum | | 19 | 18 | 18 |
| | Maximum | | 69 | 68 | 69 |
| Sex | Male | n (%) | 22 (52.4%) | 10 (25.6%) | 32 (39.5%) |
| | Female | n (%) | 20 (47.6%) | 29 (74.4%) | 49 (60.5%) |
| Employ- | No | n (%) | 19 (45.2%) | 18 (46.2%) | 37 (45.7%) |
| ment | Yes | n (%) | 23 (54.8%) | 21 (53.8%) | 44 (54.3%) |
| Disease | Mean | | 8.9 | 16.9 | 12.8 |
| duration, | Standard | | 5.28 | 7.54 | 7.60 |
| months | deviation | | | | |
| | Median | | 6.0 | 18.0 | 12.0 |
| | Minimum | | 3 | 3 | 3 |
| | Maximum | | 18 | 24 | 24 |
| Marriage | No | n (%) | 16 (38.1%) | 21 (53.8%) | 37 (45.7%) |
| status | Yes | n (%) | 26 (61.9%) | 18 (46.2%) | 44 (54.3%) |
| Children | No | n (%) | 17 (40.5%) | 21 (53.8%) | 38 (46.9%) |
| | Yes | n (%) | 25 (59.5%) | 18 (46.2%) | 43 (53.1%) |

TABLE 2
Results of statistical analysis of HADS_T assessments of changes from baseline
(V1) using a paired t-test for the evaluation visits within each patient group.

| Paired differences | | | | | | | | |
|---|---|---|---|---|---|---|---|---|
| | | | Std. | 95% confidence | | | | |
| | | Std. | Error | interval | | | | Sig. (2- |
| | Mean | Deviation | Mean | Lower | Upper | t | df | tailed) |
| Group 1 | | | | | | | | |
| HADS-T, V3 − | −4.595 | 3.379 | .521 | −5.648 | −3.542 | −8.813 | 41 | .000 |
| HADS-T, V1 | | | | | | | | |
| HADS-T, V6 − | −11.214 | 3.440 | .531 | −12.286 | −10.142 | −21.129 | 41 | .000 |
| HADS-T, V1 | | | | | | | | |
| Group 2 | | | | | | | | |
| HADS-T, V3 − | −5.692 | 1.749 | .280 | −6.259 | −5.125 | −20.319 | 38 | .000 |
| HADS-T, V1 | | | | | | | | |
| HADS-T, V6 − | −11.436 | 2.882 | .461 | −12.370 | −10.502 | −24.782 | 38 | .000 |
| HADS-T, V1 | | | | | | | | |

TABLE 3
Changes in CGI-I Scale during treatment

| | | Group | | | | | |
|---|---|---|---|---|---|---|---|
| | | «Pure PD» | | «Comorbid PD» | | Total | |
| | | (n = 42) | | (n = 39) | | (n = 81) | |
| | | n | % | n | % | n | % |
| CGI-I, V2 | Marked | 5 | 11.9% | 5 | 12.8% | 10 | 12.3% |
| | improvement | | | | | | |
| | Minimal | 20 | 47.6% | 24 | 61.5% | 44 | 54.3% |
| | improvement | | | | | | |
| | No changes | 15 | 35.7% | 10 | 25.6% | 25 | 30.9% |
| | Minimal | 1 | 2.4% | 0 | .0% | 1 | 1.2% |
| | deterioration | | | | | | |
| | Marked | 1 | 2.4% | 0 | .0% | 1 | 1.2% |
| | deterioration | | | | | | |
| | Overall | 42 | 100.0% | 39 | 100.0% | 81 | 100.0% |
| CGI-I, V3 | Marked | 17 | 40.5% | 4 | 10.3% | 21 | 25.9% |
| | improvement | | | | | | |
| | Minimal | 23 | 54.8% | 27 | 69.2% | 50 | 61.7% |
| | improvement | | | | | | |
| | No changes | 2 | 4.8% | 8 | 20.5% | 10 | 12.3% |
| | Overall | 42 | 100.0% | 39 | 100.0% | 81 | 100.0% |
| CGI-I, V4 | Marked | 34 | 81.0% | 10 | 25.6% | 44 | 54.3% |
| | improvement | | | | | | |
| | Minimal | 8 | 19.0% | 25 | 64.1% | 33 | 40.7% |
| | improvement | | | | | | |
| | No changes | 0 | .0% | 4 | 10.3% | 4 | 4.9% |
| | Overall | 42 | 100.0% | 39 | 100.0% | 81 | 100.0% |
| CGI-I, V5 | Very marked | 2 | 4.8% | 0 | .0% | 2 | 2.5% |
| | improvement | | | | | | |
| | Marked | 40 | 95.2% | 24 | 61.5% | 64 | 79.0% |
| | improvement | | | | | | |
| | Minimal | 0 | .0% | 15 | 38.5% | 15 | 18.5% |
| | improvement | | | | | | |
| | Overall | 42 | 100.0% | 39 | 100.0% | 81 | 100.0% |
| CGI-1, V6 | Very marked | 22 | 52.4% | 5 | 12.8% | 27 | 33.3% |
| | improvement | | | | | | |
| | Marked | 20 | 47.6% | 34 | 87.2% | 54 | 66.7% |
| | improvement | | | | | | |
| | Overall | 42 | 100.0% | 39 | 100.0% | 81 | 100.0% |

TABLE 4
Changes in CGI-S Scale during treatment

| | | Group | | | | | |
|---|---|---|---|---|---|---|---|
| | | «Pure PD» | | «Comorbid PD» | | Total | |
| | | (n = 42) | | (n = 39) | | (n = 81) | |
| | | n | % | n | % | n | % |
| CGI-S, V1 | Moderately expressed disease | 4 | 10.3% | 1 | 2.4% | 5 | 6.2% |
| | Significantly expressed disease | 34 | 87.2% | 38 | 90.5% | 72 | 88.9% |
| | Serious disease | 1 | 2.6% | 3 | 7.1% | 4 | 4.9% |
| | Total | 39 | 100.0% | 42 | 100.0% | 81 | 100.0% |
| CGI-S, V2 | Moderately expressed disease | 19 | 48.7% | 1 | 2.4% | 20 | 24.7% |
| | Significantly expressed disease | 20 | 51.3% | 39 | 92.9% | 59 | 72.8% |
| | Serious disease | 0 | .0% | 2 | 4.8% | 2 | 2.5% |
| | Total | 39 | 100.0% | 42 | 100.0% | 81 | 100.0% |
| CGI-S, V3 | Moderately expressed disease | 19 | 48.7% | 5 | 11.9% | 24 | 29.6% |
| | Significantly expressed disease | 20 | 51.3% | 37 | 88.1% | 57 | 70.4% |
| | Total | 39 | 100.0% | 42 | 100.0% | 81 | 100.0% |
| CGI-S, V4 | Weakly expressed disease | 0 | .0% | 20 | 47.6% | 20 | 24.7% |
| | Moderately expressed disease | 20 | 51.3% | 22 | 52.4% | 42 | 51.9% |
| | Significantly expressed disease | 19 | 48.7% | 0 | .0% | 19 | 23.5% |
| | Total | 39 | 100.0% | 42 | 100.0% | 81 | 100.0% |
| CGI-S, V5 | Borderline state | 0 | .0% | 6 | 14.3% | 6 | 7.4% |
| | Weakly expressed disease | 1 | 2.6% | 36 | 85.7% | 37 | 45.7% |
| | Moderately expressed disease | 37 | 94.9% | 0 | .0% | 37 | 45.7% |
| | Significantly expressed disease | 1 | 2.6% | 0 | .0% | 1 | 1.2% |
| | Total | 39 | 100.0% | 42 | 100.0% | 81 | 100.0% |
| CGI-S, V6 | Normal state | 0 | .0% | 5 | 11.9% | 5 | 6.2% |
| | Borderline state | 32 | 82.1% | 37 | 88.1% | 69 | 85.2% |
| | Weakly expressed disease | 7 | 17.9% | 0 | .0% | 7 | 8.6% |
| | Total | 39 | 100.0% | 42 | 100.0% | 81 | 100.0% |

TABLE 5
Descriptive statistics for the Zung Self-Rating Anxiety Scale (SAS) and changes
compared with baseline (V1) for the evaluation visits and patient groups

| | Paired Differences | | | | | | | | |
|---|---|---|---|---|---|---|---|---|---|
| | | | | | 95% confidence | | | | |
| | | | | Std. | interval of | | | | |
| | | | Std. | Error | differences | | | | Sig. (2- |
| | | Mean | Deviation | Mean | Lower | Upper | t | df | tailed) |
| Group 1 | | | | | | | | | |
| Pair 1 | Zung after 1 | −36.131 | 3.205 | .495 | −37.130 | −35.132 | −73.048 | 41 | .000 |
| | month, V1 | | | | | | | | |
| Pair 2 | Zung after 6 | −38.214 | 4.049 | .625 | −39.476 | −36.952 | −61.158 | 41 | .000 |
| | months, V1 | | | | | | | | |
| Group 2 | | | | | | | | | |
| Pair 1 | Zung after 1 | −17.308 | 7.508 | 1.202 | −19.742 | −14.874 | −14.395 | 38 | .000 |
| | month, V1 | | | | | | | | |
| Pair 2 | Zung after 6 | −16.154 | 7.562 | 1.211 | −18.605 | −13.702 | −13.340 | 38 | .000 |
| | months, V1 | | | | | | | | |

TABLE 6
Changes in the frequency of major and minor panic attacks (per month)

| Group | | |
|---|---|---|
| | «Comorbid | |
| «Pure PD» | PD» | Total |
| (n = 42) | (n = 39) | (n = 81) |

| | | | | |
|---|---|---|---|---|
| The frequency of | n | 42 | 39 | 81 |
| major panic attacks | Mean | 7.7 | 11.7 | 9.6 |
| before treatment, | Standard | 7.85 | 8.27 | 8.24 |
| times/month, V1 | Deviation | | | |
| | Percentile 25 | 1.0 | 4.0 | 2.0 |
| | Median | 3.0 | 12.0 | 7.0 |
| | Percentile 75 | 16.0 | 16.0 | 16.0 |
| | Minimum | 1 | 1 | 1 |
| | Maximum | 24 | 28 | 28 |
| The frequency of | n | 42 | 39 | 81 |
| major panic attacks | Mean | .0 | .0 | .0 |
| 6 months after | Standard | .00 | .00 | .00 |
| treatment | Deviation | | | |
| | Percentile 25 | .0 | .0 | .0 |
| | Median | .0 | .0 | .0 |
| | Percentile 75 | .0 | .0 | .0 |
| | Minimum | 0 | 0 | 0 |
| | Maximum | 0 | 0 | 0 |
| The frequency of | n | 42 | 39 | 81 |
| minor panic attacks | Mean | 44.8 | 41.7 | 43.3 |
| before treatment, | Standard | 16.18 | 15.29 | 15.73 |
| times/month, V1 | Deviation | | | |
| | Percentile 25 | 28.0 | 28.0 | 28.0 |
| | Median | 56.0 | 56.0 | 56.0 |
| | Percentile 75 | 56.0 | 56.0 | 56.0 |
| | Minimum | 4 | 4 | 4 |
| | Maximum | 84 | 56 | 84 |
| The frequency of mior | n | 42 | 39 | 81 |
| panic attacks 6 months | Mean | .3 | 1.0 | .6 |
| after treatment, | Standard | .46 | 2.64 | 1.89 |
| times/month | Deviation | | | |
| | Percentile 25 | .0 | .0 | .0 |
| | Median | .0 | .0 | .0 |
| | Percentile 75 | 1.0 | 1.0 | 1.0 |
| | Minimum | 0 | 0 | 0 |
| | Maximum | 1 | 16 | 16 |
| Changes Minor Panic | n | 42 | 39 | 81 |
| Attacs_V6_V1 | Mean | −44.5 | −40.7 | −42.7 |
| | Standard | 16.19 | 15.12 | 15.70 |
| | Deviation | | | |
| | Percentile 25 | −56.0 | −56.0 | −56.0 |
| | Median | −55.0 | −40.0 | −55.0 |
| | Percentile 75 | −28.0 | −27.0 | −28.0 |
| | Minimum | −84.00 | −56.00 | −84.00 |
| | Maximum | −4.00 | −4.00 | −4.00 |
| Changes_Major Panic | n | 42 | 39 | 81 |
| Attacs_V6_V1 | Mean | −7.7 | −11.7 | −9.6 |
| | Standard | 7.85 | 8.27 | 8.24 |
| | Deviation | | | |
| | Percentile 25 | −16.0 | −16.0 | −16.0 |
| | Median | −3.0 | −12.0 | −7.0 |
| | Percentile 75 | −1.0 | −4.0 | −2.0 |
| | Minimum | −24.00 | −28.00 | −28.00 |
| | Maximum | −1.00 | −1.00 | −1.00 |

### REFERENCES

1. Cullen, S. C. and E. G. Gross, The anesthetic properties of xenon in animals and human beings, with additional observations on krypton. Science, 1951. 113(2942): p. 580-2.
2. Dickinson, R. and N. P. Franks, Bench-to-bedside review: Molecular pharmacology and clinical use of inert gases in anesthesia and neuroprotection. Crit Care. 14(4): p. 229.
3. Adolph, O. and G. Froeba, In Vivo Measurement in Pigs of Wash-In Kinetics of Xenon at its Site of Action. Curr Clin Pharmacol, 2016.

1-25: (canceled)

26. A gas composition useful for prevention or treating an anxiety disorder in a patient consisting essentially of: 15-30% xenon and 70-85% oxygen.

27. The gas composition of claim 1, wherein said gas composition comprises 30% xenon and 70% oxygen.

28. A method of preventing or treating an anxiety in a patient in need comprising the steps of: a) selecting a patient who is diagnosed with an anxiety disorder; and b) administering to said patient one or more treatments of a composition comprising at least 5-40% xenon, and the remaining 60-95% of oxygen either prior to or during an anxious episode.

29. The method of claim 28, wherein said composition comprises 30% xenon and 70% oxygen.

30. The method of claim 28, wherein said composition is administered in a volume of 3-7 liters per treatment.

31. The method of claim 30, wherein the xenon is inhaled by the patient for between 2.5 to 4 minutes per treatment.

32. The method of claim 31, wherein the patient holds their breath for 5 to 10 seconds after an inhalation.

33. The method of claim 32, wherein the composition comprises xenon and oxygen, and lacks nitrogen and air, and is initially administered at 15% xenon/85% oxygen and escalated to 30% xenon/70% oxygen with titration increments of 5% per additional treatment.

34. The method of claim 33, wherein the patient is administered the xenon-oxygen composition daily for three days and then every other day.

35. The method of claim 33, wherein administration of said composition occurs between once per day to once every two weeks.

36. The method of claim 28, wherein said administration of said composition occurs as the patient feels the onset on the anxiety disorder.

37. The method of claim 28, wherein said administration of said composition is performed at a concentration and frequency to reduce neuroinflammation.
